(19) United States
(12) Patent Application Publication
(10) Pub. No.: US 2022/0203068 A1
(43) Pub. Date: Jun. 30, 2022

(54) Multilumen Body for a Medical Device

(71) Applicant: BIOTRONIK SE & Co. KG, Berlin (DE)

(72) Inventors: Stephan ZEDLER, Berlin (DE); Frank KLEIN, Berlin (DE); Dominik ZIMPEL, Berlin (DE); Tobias MUELLER, Berlin (DE)

(73) Assignee: BIOTRONIK SE & Co. KG, Berlin (DE)

(21) Appl. No.: 17/609,648

(22) PCT Filed: May 5, 2020
(86) PCT No.: PCT/EP2020/062355

§ 371 Date: Nov. 8, 2021

(30) Foreign Application Priority Data
May 10, 2019 (DE) 10 2019 112 248.1

(51) Int. Cl. A61M 25/00

(52) U.S. Cl. CPC A61M 25/0045; A61M 25/0032; B29L 2031/7542; A61M 2025/006; A61M 2025/0062

(57) ABSTRACT

A multilumen body for a medical device, comprising a first tubular element and a second tubular element, which is arranged in the first tubular element and which is in contact with the first tubular element at least in sections and movable relative to the first tubular element, an inner surface of the first tubular element having a first profile and/or an outer surface of the second tubular element having a second profile so that the contact surface between the first tubular element and the second tubular element is smaller than without the first profile and/or without the second profile. The multilumen body is characterized in that the first profile and/or the second profile include a plurality of alternating elevations and depressions, wherein a maximum distance between two neighboring elevations is 100 μm.

### CROSS-REFERENCE TO RELATED APPLICATIONS

This application is the United States national phase under 35 U.S.C. § 371 of PCT International Patent Application No. PCT/EP2020/062355, filed on May 5, 2020, which claims the benefit of German Patent Application No. 10 2019 112 248.1, filed on May 10, 2019, the disclosures of which are hereby incorporated by reference herein in their entireties.

### TECHNICAL FIELD

The present invention relates to a multilumen body for a medical device according to the claims, to a medical device comprising such a multi-lumen body according to the claims, and to a method for producing such a multilumen body according to the claims.

### BACKGROUND

Multilumen bodies are used in a wide range of medical devices. These are utilized, for example, as the electrode for cardiac pacemakers or for neurostimulators, as well as the shaft for catheters.

U.S. Pat. No. 7,130,700 B2 describes such a multilumen body for an implantable medical device, in which an inner lumen includes a number of notches on the outer side thereof, which serve as guide grooves for electrical conductors. An outer lumen encloses these notches and, additionally, engages in individual notches of the inner lumen that do not include an electrical conductor, thereby ensuring high resistance against rotation between the inner lumen and the outer lumen.

WIPO Publication No. WO 2015/099935 A1 describes a deflectable catheter shaft, comprising an inner lumen that has a corrugated outer structure, in a longitudinal sectional view, so as to increase the deflectability of the catheter shaft. It is also provided here that an outer lumen engages in notches that are formed by the corrugated structure of the inner lumen, so as to ensure high resistance against rotation between the outer lumen and the inner lumen, while ensuring good deflectability.

The problem with these and other approaches from the prior art is that comparatively large friction between an inner lumen and an outer lumen has to be overcome when the inner lumen is being pushed into the outer lumen. This makes it more difficult to handle such bodies, and complicates a fine adjustment of a relative position between an inner lumen and an outer lumen.

During the use of a catheter, the implantation of an electrode, or also during the later operation of an implantable electrode, these components, which, as described above, are typically designed as multilumen bodies, are regularly deflected. As a result, increasing mechanical stresses form from the inside out. The greatest mechanical stresses thus form in the outermost layer of such a multilumen body.

When this multilumen body additionally comes in contact with aggressive media, such as blood, a chemical load is added to the mechanical load. This can consequently cause stress cracks to form, whereby exterior media, such as blood, can enter the interior of such a multilumen body.

Various solutions are known from the prior art for preventing such stress crack formation. For example, the mutually contacting surfaces of individual lumina are coated with silicone compounds so as to achieve enhanced movability of the individual layers or lumina. In addition, a coating using talcum is also known. Such coatings, however, are comparatively complicated to apply and, in turn, are not necessarily chemically stable over an extended period of time.

The present disclosure is directed toward overcoming one or more of the above-mentioned problems, though not necessarily limited to embodiments that do.

### SUMMARY

It is an object of the present invention to overcome the disadvantages known from the prior art, and to provide a multilumen body in which the risk of stress crack formation is reduced compared to the non-coated bodies known from the prior art, wherein the production of this body is simplified compared to bodies comprising coatings of individual lumina.

At least this object is achieved by a multilumen body for a medical device having the features of claim 1. A multilumen body shall be understood to mean a body comprising at least two lumina. However, in principle, the body can comprise an arbitrary larger number of lumina, for example 3, 4, 5, 6, 7, 8, 9 or 10 lumina.

Such a multilumen body comprises a first tubular element and a second tubular element. The second tubular element is arranged in the first tubular element and is in contact with the first tubular element at least in sections. In addition, it is movable relative to the first tubular element. An inner surface of the first tubular element has a first profile. As an alternative or in addition, an outer surface of the second tubular element has a second profile. Since the inner surface of the first tubular element and the outer surface of the second tubular element are in contact, at least in sections, and thus form a contact surface, a reduction in this contact surface is achieved by the first profile and/or the second profile compared to a contact surface without such profiles.

According to the present invention, the multilumen body is characterized in that the first profile and/or the second profile include a plurality of alternating elevations (also referred to as peaks) and depressions (also referred to as valleys), wherein a maximum distance between two neighboring elevations is 100 μm. This means that the depressions formed between the elevations have a maximum width of 100 μm. This width is not sufficient to guide an electrical conductor in a corresponding depression. Typically, electrical conductors have an outside diameter of approximately 140 μm with insulation, and of 120 μm without insulation. While technical deliberations were already made in the past for producing conductors having even smaller dimensions, these could not be used in a conceivable multilumen body since these would have insufficient mechanical stability.

The presently claimed multilumen body is thus characterized in that the profile formed between the first tubular element (which forms a first lumen) and the second tubular element (which forms a second lumen) has such a fine structure that it is not suitable for guiding an electrical conductor, and in particular not an electrical conductor having mechanical stability that is sufficiently large to ensure safe use of such an electrical conductor in a deflectable multilumen body.

Surprisingly, it has been shown that such a fine profile achieves a significant reduction in the frictional forces that occur between the first tubular element and the second tubular element during a relative movement of the two tubular elements with respect to one another. As a result of this significant reduction in the frictional forces, the second tubular element is able to move comparatively easily relative to the first tubular element, for example when the multilumen body is being deflected. In this way, the introduction of mechanical stresses into the (outer) first tubular element is prevented, whereby the risk of stress crack formation is significantly reduced. As a result, not only the durability of the first tubular element increases, but also the durability of the multilumen body as a whole, since the ingress of external media into an interior region of the multilumen body is effectively prevented, even after a long usage duration.

The profile or structure of the first tubular element and/or of the second tubular element thus ensure, entirely without chemical additives or coatings, that a significant reduction in friction is achieved, which ensures an overall extended service life of the multilumen body. This purely mechanical or physical option of extending the service life of such multilumen bodies is superior to the approaches known from the prior art. The reason is that no additional method step is required, in which a coating would be applied to a surface of one of the tubular elements. Rather, the profile can be introduced directly into the corresponding surface during the production process (for example, within the scope of an extrusion process). Moreover, no additional materials are required, which lowers the overall production costs of the multilumen body.

In one variant, the distance between two neighboring elevations is in a range between 5 μm and 100 μm, in particular between 10 μm and 90 μm, in particular between 20 μm and 80 μm, in particular between 30 μm and 70 μm, in particular between 35 μm and 60 μm, and in particular between 40 μm and 50 μm.

Typically, extrusion tools for producing profiled tubular elements are produced by way of electrical discharge machining, such as wire and die sink EDM. As an alternative, other traditional metal working methods may also be employed. However, even when using electrical discharge machining, it is not possible to manufacture extrusion tools that are suitable for producing profiled tubular elements in which the distance between two neighboring elevations is smaller than 300 μm. If structures are to be produced in which the distance between two neighboring elevations is smaller than 300 μm, other ways thus have to be used to manufacture the extrusion tools. A laser can be used to achieve finer structures at the extrusion tool. However, a tool and die maker would not use a laser in this situation, given the massive design of typical extrusion tools, since the production times would thus be too long. For this reason, the inventors provided an additional aperture in the extrusion tool, into which the fine structure was introduced by way of a laser. Typically, fine apertures are not used in extrusion tools for reasons related to wear.

In principle, the first profile and/or the second profile can have any arbitrary design. These can be configured identically or differently. For example, these can be configured in the form of a regular pattern or in the form of an irregular pattern. For example, the profile can be configured in the form of a rectangular pattern, a wave pattern or a circular pattern. The pattern can be a recurring pattern. It is also conceivable that, when two profiles are present, one of the two profiles has a regular pattern, and the other of the two profiles has an irregular pattern.

In one variant, the first profile and/or the second profile have a corrugated shape in the cross-section of the multilumen body. Only one profile is known from WIPO Publication No. WO 2015/099935 A1, which has a corrugated shape in the longitudinal section view of the catheter described there. However, the catheter described there does not have a corrugated profile in the cross-sectional view. This would be useless for the purpose pursued by the international patent application (namely that of easier deflectability of the catheter described therein).

When the first profile and/or the second profile have a corrugated shape, in the cross-sectional view, the elevations of the corrugated profile typically make contact with the respective other tubular element, while the depressions do not contribute to the contact surface, and thus cause a decrease in the contact surface.

The elevations and depressions of the first profile and/or the second profile can, for example, be configured in the form of individual protuberances and regions located between the protuberances. In this embodiment, there are a plurality of elevations of the profile which are not connected.

In another embodiment, the elevations and depressions of the first profile and/or the elevations and depressions of the second profile extend from a proximal end of the multilumen body to a distal end of the multilumen body. This means that, in this embodiment, the respective elevations are structures extending in an elongated manner, from the proximal end of the multilumen body to the distal end of the multilumen body. The depressions then have the shape of flutes or grooves, which are arranged between two neighboring elevations. In this embodiment as well, it does not adversely impact the technical effect of friction reduction if individual interruptions are present within the elevations and/or depressions of the particular profile. Even if such interruptions are present, a significant reduction in the frictional forces that act between the first tubular element and the second tubular element during a relative movement is achieved. Such individual interruptions are thus also covered by the term, when speaking of an extension of the elevations and depressions from the proximal end to the distal end of the multilumen body.

In one embodiment, the elevations and depressions of the first profile and/or the elevations and depressions of the second profile extend substantially in a longitudinal direction of the multilumen body. In other words, these extend along a longitudinal extension direction of the multilumen body. It is not absolutely necessary for these to be exactly aligned with the longitudinal extension direction of the body. Rather, deviations from the longitudinal extension direction are conceivable, provided a basic elongated extension direction is present. Specifically, the elevations and depressions can extend, for example, in a linear or helical (spiral-shaped) manner along the longitudinal extension direction. Such an embodiment can be achieved particularly easily by way of an extrusion process using an appropriately configured tool, wherein a rotation of the tool takes place during the extrusion process with a helical orientation of the elevations and depressions. This rotation can, in particular, occur continuously in one direction, so as to achieve a (slightly) continuously winding orientation of the elevations and depressions about a longitudinal axis, which runs along the longitudinal extension direction of the multilumen body.

A space or a cavity is formed between the first tubular element and the second tubular element, in particular in the region of the depressions of the at least one profile. In one embodiment, the space or cavity is filled with a gas. The gas can be a gas mixture. The gas or gas mixture can be one or more of the gases that are nitrogen, oxygen, carbon dioxide, and/or one or more noble gases from the group consisting of neon, argon, krypton or xenon. When the multilumen body is used as intended, the gas or gas mixture does not just remain temporarily in said space or cavity, even in the implanted state. As a result of the gas, the sliding properties between the first tubular element and the second tubular element are the same everywhere along the multilumen body. Moreover, the best possible electrical insulating effect is achieved by the gas.

In one variant, the second tubular element comprises at least one inner lumen, which is provided and configured to accommodate an electrical conductor. In such a case, the multilumen body can be used particularly well as an electrode for a medical device. At the same time, the second tubular element serves as an insulator for the electrical conductor, which is guided through the inner lumen. It is possible and contemplated that the second tubular element comprises 2, 3, 4, 5, 6, 7 or 8 lumina, for example, wherein an electrical conductor can be guided through individual or each of these inner lumina.

In one embodiment, the first tubular element and/or the second tubular element are made of at least one polymer. It is possible to use the same polymer, or different polymers, for the first tubular element and the second tubular element. In one variant, the first tubular element and/or the second tubular element comprise at least one polymer, which is selected from the group consisting of polyurethanes (PU), polyester urethanes (PEU), polyether urethanes (PEEU), polycarbonate urethanes (PCU), silicone-based polycarbonate urethanes (PCU), polycarbonate polyurea urethanes (PCHU), polydimethylsiloxane urethanes (PSU), polyisobutylene urethanes (PIU), polyisobutylene-based copolymers (PIC), polyether block amides (PEBA, for example PEBAX), polyimides (PI), fluorinated hydrocarbons, ethylene-tetrafluoroethylene copolymer (ETFE), polytetrafluoroethylene (PTFE), tetrafluoroethylene (TFE), perfluoro (ethylene-propylenee) (FEP), perfluoroalkoxy polymers (PFA), polysulfone (PSU), polyethylene (PE), polypropylene (PP), polyamides (PA), and silicone. In one variant, the first tubular element and/or the second tubular element are made of one of the aforementioned polymers.

In one variant, the polymer is a thermoplastic material, a thermoset material or an elastomer. The majority of the polymers listed above are thermoplastics. Such thermoplastics have been proven to be particularly advantageous for the production of the first tubular element and/or of the second tubular element. It is thus provided in one variant that the first tubular element and/or the second tubular element comprise or consist of a thermoplastic material, and in particular a thermoplastic material from the aforementioned group of polymers. In particular, it is provided that the first (that is, outer) tubular element comprises or consists of such a thermoplastic material. In particular, silicone is a suitable material for the second tubular element.

Polyurethanes can have thermoplastic, elastomeric or thermoset properties. This is determined, in particular, by the degree of cross-linking of the monomers and the individual polymer chains among one another. In one variant, it is thus provided that a thermoplastic polyurethane is used as the thermoplastic. It is likewise possible to produce polyimides having thermoplastic or thermoset properties. In one variant, the polyamide to be used is a thermoplastic polyimide.

In one variant, the first tubular element comprises or is made of a thermoplastic polyurethane, while the second tubular element comprises or is made of a silicone. In particular, it is provided in this variant that the first tubular element is made of a thermoplastic polyurethane, and the second tubular element is made of a silicone.

Silicones have good electrically insulating properties, so that the second tubular element in this embodiment is particularly suitable for accommodating electrical conductors in the interior thereof (namely in an inner lumen provided therefor). One or more electrical conductors can be provided, wherein one or more inner lumina of the second tubular element can be provided for this purpose. The conductors can run in the corresponding inner lumina without further insulation since the silicone then assumes the electrical insulation of the electrical conductors. The thermoplastic polyurethane of the first tubular element, which surrounds the second tubular element made of silicone, ensures an abrasion-resistant casing of the second tubular element, and thus increases the service life compared to an embodiment in which no such outer thermoplastic layer is provided. Due to the profile of the inner surface of the first tubular element or of the outer surface of the second tubular element, good relative movement of the two elements with respect to one another is nonetheless made possible as a result of the reduced frictional force, and likewise ensures an extended service life of a multilumen body configured in this way.

If the first tubular element is made of a thermoplastic material, and in particular of a thermoplastic polyurethane, it can, in one embodiment, have a Shore hardness, measured according to DIN EN ISO 868 and/or DIN ISO 7619-1, in the range of 80 A to 75 D, in particular 90 A to 70 D, in particular 95 A to 60 D, in particular 100 A to 55 D, in particular 5 D to 50 D, in particular 10 D to 40 D, and in particular 20 D to 30 D.

If the second tubular element is made of silicone, it can, in one embodiment, have a Shore hardness, measured according to DIN EN ISO 868 and/or DIN ISO 7619-1, in the range of 30 A to 85 A, in particular 40 A to 80 A, in particular 50 A to 70 A, and in particular 60 A to 65 A.

The first profile additionally imparts increased stability to the first tubular element. Likewise, the second profile results in enhanced stability of the second tubular element. In this way, it is possible to produce each tubular element that has a profile with a lower wall thickness than in the case of a non-profiled design. As a result, the overall diameter of the multilumen body can be reduced. In one variant, the reduction of the outer diameter over comparable multilumen bodies comprising non-profiled tubular elements is 0.05 to 0.5 mm, in particular 0.1 to 0.4 mm, and in particular 0.2 to 0.3 mm.

In one embodiment, the multilumen body has both the first profile and the second profile. However, the first profile and the second profile do not engage one another in the process, but allow a rotation of the second element in the first element. In this embodiment, the first profile and the second profile are thus precisely not configured to interlock, so that a torsional force exerted on the first tubular element or on the second tubular element is precisely not transferred to the respective other element, but results in a relative rotational movement of the second element within the first element. Due to the provision of two profiles, which do not engage one another, the contact surface between the first tubular element and the second tubular element is further reduced, resulting in a further decrease of the frictional forces that occur.

In one variant, the multilumen body likewise has the first profile and the second profile. The first profile and the second profile have different orientations with respect to the multilumen body, so that a punctiform contact pattern results between the first profile and the second profile. For example, the first profile and the second profile can each be configured as helical, longitudinally extending elevations and depressions, wherein the first profile is configured to be right helical, and the second profile is configured to be left helical. The contact surface between the first tubular body (or the inner side thereof) and the second tubular element (or the outer side thereof) is then reduced even further, which results in an even further decrease of the frictional forces that occur between these two elements.

A space or a cavity is formed between the first tubular element and the second tubular element, in particular in the region of the depressions of the at least one profile. In one embodiment, this space in a proximal end region of the multilumen body and/or in a distal region of the multilumen body is filled with an adhesive. After curing, this adhesive is used to prevent the ingress of fluid from outside the multilumen body into the space between the first tubular element and the second tubular element. Such a bonded joint between the first tubular element and the second tubular element in the proximal and/or distal end region of the multilumen body does not impair the service life of the multilumen body as a whole. The reason is that the sections of the first tubular element and of the second tubular element located between the proximal end region and the distal end region can still be moved relative to one another. In this way, mechanical stresses are prevented from building between the two elements, despite such an adhesive bond of the distal and/or proximal end region.

As a result of the profile of the first tubular element and/or of the second tubular element, it is additionally possible to better check visually whether, and across what range, adhesive has already penetrated into the space between the first tubular element and the second tubular element. The profile thus facilitates the production of a multilumen body, in the proximal end region and/or distal end region of which a space formed between the first tubular element and the second tubular element is closed with an adhesive.

The proximal end region shall be understood to mean the region of the multilumen body that extends from the proximal end of the body across a maximum of 10%, in particular a maximum of 9%, in particular a maximum of 8%, in particular a maximum of 7%, in particular a maximum of 6%, in particular a maximum of 5%, in particular a maximum of 4%, in particular a maximum of 3%, in particular a maximum of 2%, and in particular a maximum of 1% of the total length of the multilumen body in the longitudinal extension direction to the distal end of the multilumen body. Similarly, the expression “distal end region” shall be understood to mean the region of the multilumen body that extends from the distal end of the body across a maximum of 10%, in particular a maximum of 9%, in particular a maximum of 8%, in particular a maximum of 7%, in particular a maximum of 6%, in particular a maximum of 5%, in particular a maximum of 4%, in particular a maximum of 3%, in particular a maximum of 2%, and in particular a maximum of 1% of the total length of the multilumen body in the longitudinal extension direction to the proximal end of the multilumen body.

The adhesive joining between the first tubular element and the second tubular element in the proximal and/or distal end region of the multilumen body takes place across a length of 0.5 mm to 30 mm, in particular across a length of 1 mm to 15 mm, and in particular across a length of 1 to 5 mm.

In one variant, the multilumen body is configured as a shaft of a catheter and/or is used as such a catheter shaft.

In another variant, the multilumen body is configured as an implantable electrode and/or is used as such an implantable electrode. This may be, for example, an implantable electrode for a cardiac pacemaker, for a cardioverter/defibrillator or a neurostimulator.

One aspect of the present invention relates to a medical device comprising a multilumen body according to the above description. In this way, it is possible to directly apply the effects that were described above with respect to the multilumen body to the medical device equipped with such a multilumen body.

In one variant, the medical device is an implantable device for stimulating the human or animal heart. Examples of such stimulation devices are an implantable cardiac pacemaker and an implantable cardioverter/defibrillator. In this case, the multilumen body is an implantable electrode of this implantable device.

In another variant, the medical device is an implantable device for stimulating the nerves of the human or animal body. An example of such a stimulation device is an implantable neurostimulator for stimulating the spinal cord or for stimulating the vagus nerve. In this case, the multilumen body is an implantable electrode of this implantable device.

One aspect of the present invention relates to a method for producing a multilumen body according to the above description. As shown, such a multilumen body comprises a first tubular element and a second tubular element arranged in the first tubular element. The second tubular element makes contact with the first tubular element at least in sections and can be moved relative to the first tubular element. An inner surface of the first tubular element is provided with a first profile. As an alternative or in addition, an outer surface of the second tubular element is provided with a second profile. The at least one provided profile causes the contact surface between the first tubular element and the second tubular element to be smaller than if no profile of the surfaces of the tubular elements were provided.

According to the present invention, the method is characterized in that the first tubular element and/or the second tubular element are produced by way of extrusion using an extrusion tool. The extrusion tool is designed so as to introduce the first profile into the first tubular element and/or the second profile into the second tubular element during the extrusion process. The first profile and/or the second profile include a plurality of alternating elevations and depressions, wherein a maximum distance between two neighboring elevations is 100 μm.

For example, an extrusion tool having a star-shaped or corrugated cross-section is a suitable extrusion tool, wherein the points of the star or the peaks of the corrugation represent elevations, and the interposed regions represent depressions, in the profile of a tubular element produced by way of this tool.

In one method variant, the extrusion tool is rotated during the extrusion process about an axis extending in the extrusion direction so as to achieve a helical orientation of elevations and depressions in the extruded tubular element.

In an alternative production method, the first profile is introduced into the first tubular element and/or the second profile is introduced into the second tubular element by way of etching or by way of a lithography process. Using such methods, even finer structures can typically be implemented for the profile than if the profile is produced by way of an extrusion process. However, such etching or such a lithography process necessitates an additional method step, whereby the entire production method becomes slightly more complex again.

All variants and embodiments that were described in connection with the claimed multilumen body can be arbitrarily combined with one another and applied individually, or in any combination, to the described medical device and the described production method. Similarly, variants and embodiments of the medical device can be applied individually, or in any combination, to the multilumen body or the production method. Finally, variants of the production method can be applied individually, or in any combination, to the multilumen body or the medical device.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

Further details of aspects of the present invention will be described hereafter in more detail based on exemplary embodiments and figures. In the drawings:

FIG. 1 shows an exemplary embodiment of a multilumen body; and

FIG. 2 shows a cross-section through the multilumen body of FIG. 1.

### DETAILED DESCRIPTION

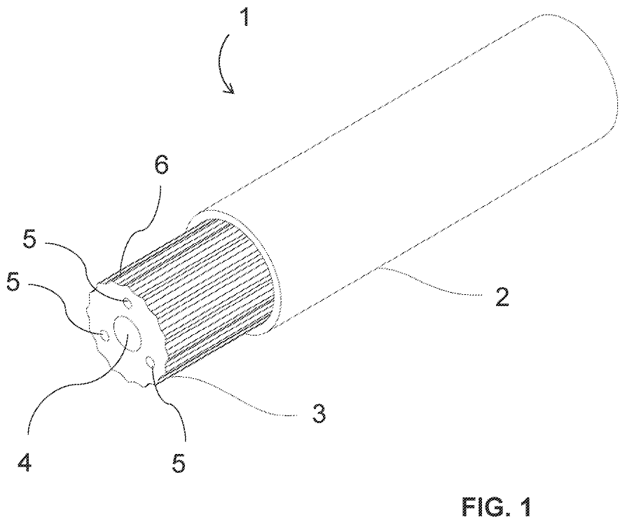

FIG. 1 shows a section of an insulating tube 1 for a cardiac pacemaker electrode, which comprises an outer cover tube 2 and a silicone tube 3 arranged in the outer cover tube 2. The outer cover tube 2 serves as a first tubular element, whereas the inner silicone tube 3 serves as a second tubular element. The cover tube 2 comprises a lumen in the interior thereof, in which the silicone tube 3 is accommodated. The silicone tube 3, in turn, comprises a central inner lumen 4 and three peripheral inner lumina 5. The insulating tube 1 is consequently a multilumen body.

An outer side of the silicone tube 3 is in close contact with an inner side of the cover tube 2. So as to reduce the size of the contact surface between the silicone tube 3 and the cover tube 2, the silicone tube 3 has a profile 6, which serves as a second profile, on the outer surface thereof. In the exemplary embodiment shown in FIG. 1, the inner surface of the cover tube 2, which is oriented toward the outer surface of the silicone tube 3, is not profiled, but has a smooth design.

So as to produce the insulating tube 1, the cover tube 2 and the silicone tube 3 are extruded independently of one another. Thereafter, the tubes thus produced are pushed inside one another. This kind of pushing inside one another is typically comparatively difficult since high friction results between the tubes to be pushed inside one another.

As a result of the profile 6, however, both the static friction and the sliding friction between the cover tube 2 and the silicone tube 3 are significantly reduced, so that the silicone tube 3 can be easily pushed into the cover tube 2, or the cover tube 2 can be easily pulled over the silicone tube 3.

The central inner lumen 4 of the silicone tube 3 is used to accommodate a guide wire or stylet so as to introduce the insulating tube 1 into a human body or an animal body (in particular when it was already finished to form a pacemaker electrode). The peripheral inner lumina 5 are used to accommodate a respective electrical conductor, wherein such an electrical conductor does not necessarily have to be provided in each of the inner lumina 5.

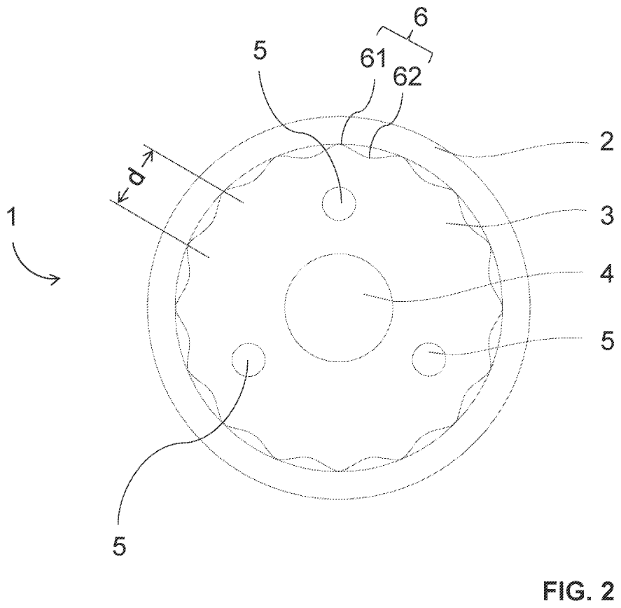

FIG. 2 shows a cross-section through the insulating tube 1 of FIG. 1, wherein like elements are denoted by like reference numerals.

The profile 6 is even more apparent in the cross-sectional illustration of FIG. 2, which in the exemplary embodiment of FIG. 2 has a corrugated design. It encompasses a plurality of alternating peaks 61, which serve as elevations, and valleys 62, which serve as depressions. A distance d between two neighboring peaks 61 of the profile 6 is 100 μm in the exemplary embodiment of FIG. 2. It must be taken into consideration in the process that FIG. 2 is not implemented to scale. Rather, the profile 6 is illustrated in enlarged form so as to be better visible.

This distance of 100 μm is not suitable for accommodating an electrical conductor within a valley 62 of the profile 6. Rather, only the peripheral inner lumina 5 are used to accommodate electrical conductors within the silicone tube 3.

### Exemplary Embodiment: Reduction of Friction

First, as a comparative example, an inner silicone tube without a profiled outer surface, having an outside diameter of 2.55 mm, was used, and covered with an outer cover tube. This cover tube had an inside diameter of 2.55 mm and an outside diameter of 2.65 mm. The cover tube was made of a thermoplastic polyurethane. Both the static friction force and the sliding friction force were ascertained, which have to be overcome to pull the cover tube over the silicone tube.

Thereafter, the experiment was repeated with a silicone tube having an outer profile. The silicone tube was made of the same material as the silicone tube of the comparison experiment. Furthermore, a cover tube made of the same material and having the same dimensions as the cover tube of the comparison experiment was used.

The profiled silicone tube likewise had a maximum outside diameter of 2.55 mm, additionally having a profile that was corrugated, in the cross-sectional view, at the outer surface thereof across the entire length thereof. The distance between two neighboring peaks of this corrugated structure was 100 μm.

The static friction force to be overcome and the sliding friction force to be overcome were also ascertained in the case of the profiled silicone tube. Thereafter, the ratio of the static friction forces for the profiled silicone tube to the non-profiled silicone tube and the ratio of the sliding friction forces for the profiled silicone tube to the non-profiled silicone tube were found.

Ultimately, it was possible to ascertain that the static friction force in the case of the profiled silicone tube was 75% to 85% lower than in the case of the non-profiled silicone tube, while the sliding friction force was approximately 65% to 75% lower than in the case of the non-profiled silicone tube. Measurements conducted on the profiled silicone tube having a maximum outside diameter of 2.55 mm and a corrugated profile, in the cross-sectional view, on the outer surface thereof, with a distance between two neighboring peaks of this corrugated structure of 32 μm, yielded comparable results.

### Exemplary Embodiment: Reduction of the Contact Surface

So as to ascertain how much the contact surface between the outer cover tube and the inner silicone tube can be reduced by profiling of the silicone tube, the insulating tubes used in the preceding exemplary embodiment were further examined. In the case of the non-profiled silicone tube, the silicone tube rested completely against the cover tube. The contact surface between the cover tube and the non-profiled silicone tube thus corresponded to the inner cross-sectional circumference of the cover tube or the outer cross-sectional circumference of the silicone tube. A diameter of 2.55 mm thus resulted in a contact line of approximately 8 mm.

The profiled silicone tube included 80 peaks and interposed valleys, which were located 100 μm away from one another. On average, each peak was in contact with the inner surface of the cover tube across a width of 23 μm. This adds up to a contact line of 1.84 mm, which corresponds to a reduction in the contact line of approximately 77%.

When the cover tube is pulled equally far over the non-profiled silicone tube and the profiled silicone tube, likewise a reduction in the contact surface between the cover tube and the silicone tube of 77% results.

Another profiled silicone tube, having a comparable diameter, included valleys that were located 32 μm away from one another. Likewise, a reduction in the contact line of approximately 77% was measured in the case of this tube.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A multilumen body for a medical device, comprising a first tubular element and a second tubular element, which is arranged in the first tubular element and which is in contact with the first tubular element at least in sections and movable relative to the first tubular element, an inner surface of the first tubular element having a first profile and/or an outer surface of the second tubular element having a second profile so that the contact surface between the first tubular element and the second tubular element is smaller than without the first profile and/or without the second profile,
wherein the first profile and/or the second profile include a plurality of alternating elevations and depressions, a maximum distance between two neighboring elevations being 100 μm.

2. The multilumen body according to claim 1, wherein the first profile and/or the second profile have a corrugated shape in the cross-section of the multilumen body.

3. The multilumen body according to claim 1, wherein the elevations and depressions of the first profile and/or the elevations and depressions of the second profile extend from a proximal end of the multilumen body to a distal end of the multilumen body.

4. A multilumen body according to claim 1, wherein the elevations and depressions of the first profile and/or the elevations and depressions of the second profile extend in a linear or helical manner along a longitudinal extension direction of the multilumen body.

5. A multilumen body according to claim 1, wherein the second tubular element comprises at least one inner lumen, which is provided and configured to accommodate an electrical conductor.

6. A multilumen body according to claim 1, wherein the first tubular element and/or the second tubular element comprise at least one polymer, which is selected from the group consisting of polyurethanes, polyester urethanes, polyether urethanes, polycarbonate urethanes, polycarbonate polyurea urethanes, polydimethylsiloxane urethanes, polyisobutylene urethanes, polyisobutylene-based copolymers, polyether block amides, polyimides, fluorinated hydrocarbons, ethylene-tetrafluoroethylene copolymer, polytetrafluoroethylene, tetrafluoroethylene, perfluoro, perfluoroalkoxy polymers, polysulfone, polyethylene, polypropylene, polyamides, and silicone.

7. A multilumen body according to claim 1, wherein the first tubular element comprises a thermoplastic material, and the second tubular element comprises silicone.

8. A multilumen body according to claim 1, wherein the multilumen body includes both the first profile and the second profile, the first profile and the second profile not engaging one another, but allowing a rotation of the second tubular element in the first tubular element.

9. A multilumen body according to claim 1, wherein the multilumen body includes both the first profile and the second profile, the first profile and the second profile having different orientations so that a punctiform contact pattern results between the first profile and the second profile.

10. A multilumen body according to claim 1, wherein an adhesive is situated in a space between the first tubular element and the second tubular element in a proximal end region of multilumen body and/or in a distal end region of the multilumen body, the adhesive preventing an ingress of a fluid from outside the multilumen body into the space between the first tubular element and the second tubular element.

11. A multilumen body according to claim 1, wherein the multilumen body is a shaft of a catheter.

12. A multilumen body according to claim 1, wherein the multilumen body is an implantable electrode.

13. A medical device, wherein a multilumen body according to claim 1.

14. The medical device according to claim 13, wherein the medical device is an implantable device for stimulating the human or animal heart, such as an implantable cardiac pacemaker or an implantable cardioverter/defibrillator, and the multilumen body is an implantable electrode of this implantable device.

15. A method for producing a multilumen body according to claim 1, comprising a first tubular element and a second tubular element, which is arranged in the first tubular element and which is in contact with the first tubular element at least in sections and movable relative to the first tubular element, an inner surface of the first tubular element having a first profile and/or an outer surface of the second tubular element having a second profile so that the contact surface between the first tubular element and the second tubular element is smaller than without the first profile and/or without the second profile, the method comprising:
producing the first tubular element and/or the second tubular element by way of extrusion using an extrusion tool, the extrusion tool being designed so as to introduce the first profile into the first tubular element and/or the second profile into the second tubular element during the extrusion process, the first profile and/or the second profile including a plurality of alternating elevations and depressions, a maximum distance between two neighboring elevations being 100 μm.
